(19) United States
(12) Patent Application Publication
(10) Pub. No.: US 2005/0171538 A1
(43) Pub. Date: Aug. 4, 2005

(54) Vertebral arthrodesis device

(76) Inventors: Frederic Sgier, Luzern (CH); Jean-Jacques Martin, Bourg En Bresse (FR)

Correspondence Address: YOUNG & THOMPSON, 745 SOUTH 23RD STREET, 2ND FLOOR, ARLINGTON, VA, 22202, US

(21) Appl. No.: 10/766,911

(22) Filed: Jan. 30, 2004

(51) Int. Cl. A61B 17/70; A61B 17/56

(52) U.S. Cl. 606/61

(57) ABSTRACT

The device includes at least one pin and parts for anchoring this pin to the vertebrae. The device includes spherical rings having a plurality of slots distributed on their peripheries. The head of each anchoring part has a cavity that may receive one of the rings with snap-on installation, and at least two lateral threaded holes. Each anchoring part includes a clamping ring presenting a central aperture in a conical form allowing the clamping ring to come into contact with the ring during tightening of the screws that are within the clamping ring.

### FIELD OF THE INVENTION

The present invention relates to a device for vertebral arthrodesis.

### DESCRIPTION OF THE RELATED PRIOR ART

Such a device comprises, as is well known, one or two staying pins for the vertebral column, designed to be positioned along the vertebrae that are to be immobilized, and parts for anchoring this or these pins to the vertebrae. These anchoring parts are generally constituted of pedicle screws or hooks whose heads delimit the receiving cavities of the pins and receive the clamping rings of a pin in these cavities.

A construction has been devised which allows the possibility of clearing a staying pin with relation to a pedicle screw and the possibility of immobilizing this pin with relation to this screw in a position in which the pin is not perpendicular to the axis of the screw. A known pedicle screw of this type presents a spherical head pierced by a hole through which the pin is designed to be engaged, this hole being threaded and receiving two nuts between which said spherical head may be tightened when the nuts are screwed on the pin.

A system of this type, outside of its manufacturing complexity, has the significant disadvantages of not being easy to position and of not ensuring a complete immobilization of the pin in a given position.

The present invention aims to remedy these essential disadvantages.

### SUMMARY OF THE INVENTION

The device to which the present invention relates comprises, in a manner that is in itself known, at least one pin designed to be positioned along the vertebrae that are to be immobilized, and the parts for anchoring this pin to the vertebrae, each anchoring part presenting a head that delimits a receiving cavity for the pin and that receives the means for tightening the pin in the said cavity, the assembly allowing the possibility of clearing the pin with relation to the anchoring part before tightening the said tightening means, and the possibility of immobilizing this pin with relation to this anchoring part in a given position, when the tightening is performed.

According to the invention,
The device comprises spherical rings in a number equal to that of the anchoring parts, each ring presenting an inside diameter allowing it to be slidingly engaged on the pin and a plurality of slots distributed on its periphery, these slots extending between the outer surface of the ring and the said inside diameter and opening alternately at one of the longitudinal openings of this inside diameter and at the other of these longitudinal openings;
The head of each anchoring part is shaped so that the said cavity that the part delimits may receive one of said rings with snap-on installation, and presents at least two lateral threaded holes opening from its proximal surface; and
Each anchoring part comprises a clamping ring shaped so that it can be positioned on said head by overlapping said ring, this clamping ring comprising lateral holes appropriate for coinciding with the threaded holes of the head and for receiving the tightening screws that may be screwed in these threaded holes, this clamping ring furthermore presenting a cone-shaped central aperture allowing the clamping ring to come into contact with the ring in the course of tightening the said screws, so that this clamping ring tightens this ring between itself and said head.

The pin may also slidingly receive the rings in a number equal to the number of anchoring parts designed to fix this pin to the vertebrae. Once these anchoring parts are positioned, the pin is shaped in the desired manner for allowing the desired correction of the vertebral column, then each ring is clipped in the cavity of each anchoring part. The various clamping rings are then positioned and tightened onto the heads of the various anchoring parts; the tightening of the rings by said lateral screws and by the inclined walls of the conical apertures of the clamping rings, allows a complete immobilization of the pin with relation to the anchoring parts to be ensured, by radial displacement of the rings, and this is possible whatever the angular orientations of the rings in the cavities of the anchoring parts or whatever the orientation of the pin with relation to the anchoring parts may be.

Preferably, the head of each anchoring part comprises at least one slot opening in the bottom of the said cavity, this gives a slight flexibility in the direction perpendicular to that according to which this cavity opens on the outside of the head; advantageously, this head comprises two lateral slots opening in the bottom of the said cavity. In this case, each slot may be inclined in the direction of the other slot so that the slots converge in the direction of the bottom of the cavity.

To be clearly understood, the invention is described again below, with reference to the attached schematic drawing showing, by way of a non-limiting example, a preferred embodiment of the device to which the invention relates.

### BRIEF DESCRIPTION OF THE DRAWINGS

FIG. 1 is a perspective view of a staying pin for the vertebral column, of a spherical ring and of a pedicle screw that comprises this device, before assembly;

FIG. 2 is a view of this pin, this ring and this screw in section according to the axis of the screw, following assembly;

FIG. 3 is a view of this pin, this ring and this screw, in section according to the axis of the screw and in a plane perpendicular to that of FIG. 2, and

FIG. 4 is a view of the device following positioning on the lumbar portion of the vertebral column.

### DETAILED DESCRIPTION OF THE PREFERRED EMBODIMENTS

FIG. 4 represents a vertebral arthrodesis device 1 comprising one or two pins 2 for staying to the vertebral column, designed to be positioned along the vertebrae 3 that are to be immobilized, and pedicle screws 4 for anchoring this or these pins 2 to the vertebrae 3.

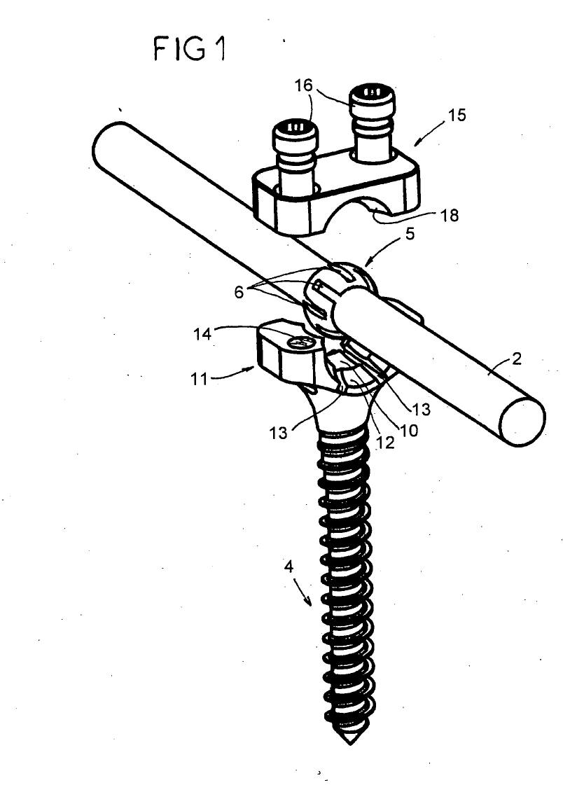

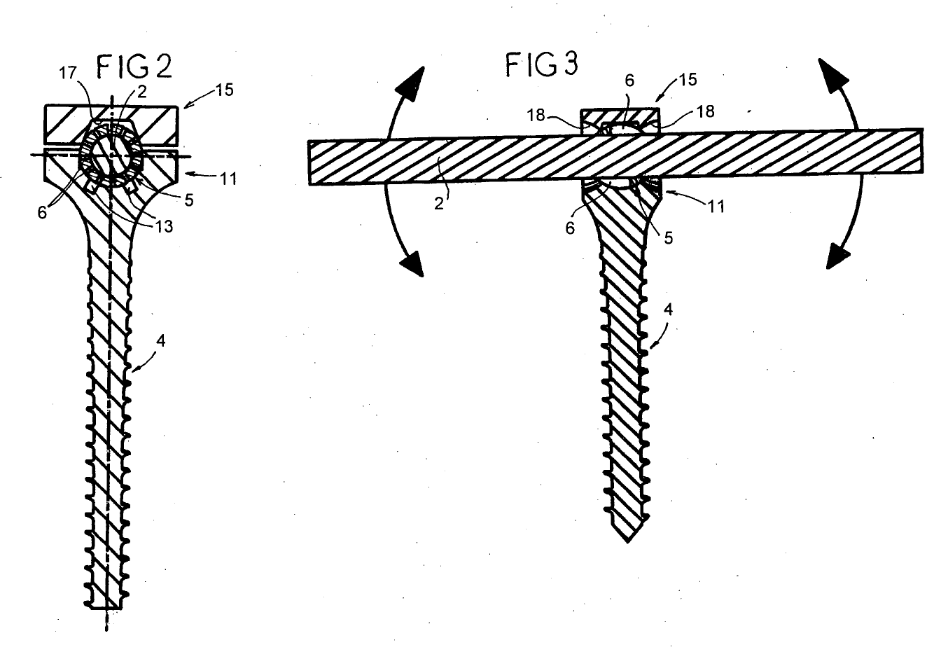

As shown in FIGS. 1 to 3, the device also comprises rings 5 of a spherical form in a number equal to that of the pedicle screws 4. FIG. 1 more particularly shows that each ring 5 presents an inside diameter allowing its sliding engagement on a pin 2 and a plurality of slots 6 distributed on its periphery. These slots 6 extend between the outer surface of the ring 5 and the said inside diameter and open alternatively at one of the longitudinal openings of this inside diameter and at the other of these longitudinal openings.

Each pin 2 is metallic and presents a cylindrical form. The pin may be curved according to the correction of the vertebral column to be performed, as well as that appearing in FIG. 4.

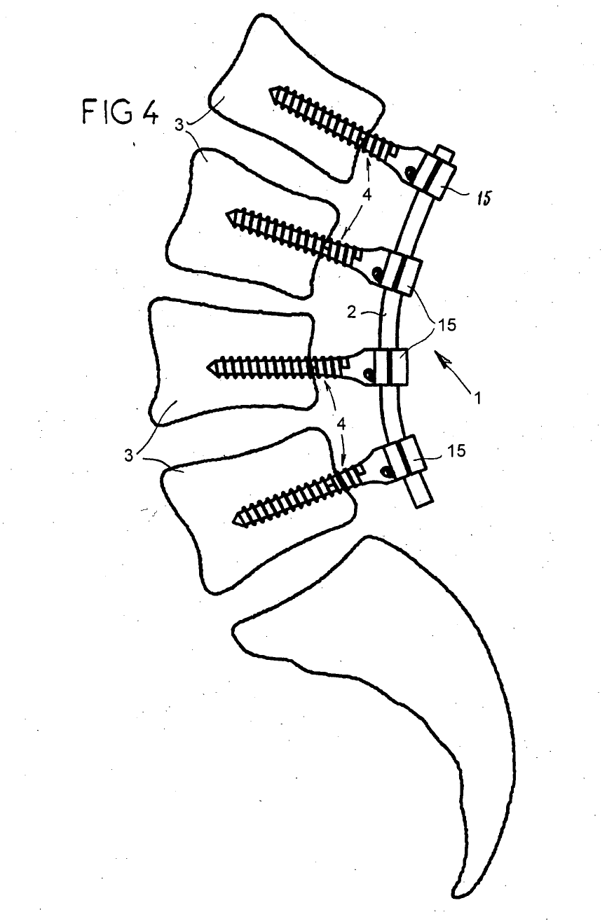

Each ring 5 is designed to be received in a cavity 10 provided in the proximal head 11 of each pedicle screw 4. This cavity 10 is delimited by a wall in the form of a segment of a hollow sphere presenting a diameter slightly smaller than that of each ring 5, so that each ring 5 may be engaged and held inside the cavity 10 by snap-on installation. The latter is bordered by two lateral undercuts 12 in the form of a segment of a hollow sphere that allow, in this snap-on installation position of the ring 5, clearance of the pin 2 with relation to the screw 4, as shown by the arrows appearing in FIG. 3.

Each head 11 also presents two slots 13 opening in the bottom of the cavity 10 and in the undercuts 12, disposed so that they are substantially radial with relation to this cavity and these undercuts. These slots 13 thus converge one toward the other in the direction of the bottom of the cavity 10; they give a slight flexibility to the head 11 in the direction perpendicular to that according to which the cavity 10 opens on the outside, to allow the aforesaid snap-on installation of the ring 5 in the cavity 10.

Furthermore each head 11 presents an enlarged form extending in a direction perpendicular to the direction according to which the cavity 10 opens on the outside of the head 11, so that the form delimits two thick lateral walls, in the proximal surfaces of which two threaded holes 14 are provided.

Each screw 4 comprises a tightening cap 15 shaped for being positioned on the head 11 by overlapping the ring 5 clipped in the cavity 10. This cap 15 comprises lateral holes appropriate for coinciding with the holes 14 and for receiving the tightening screws 16 that can be screwed in these holes 14.

Furthermore, the cap 15 presents a cone-shaped central aperture, permitting the cap to come into contact with the ring 5 in the course of tightening the screws 16, and two lateral undercuts 18 similar to the undercuts 12.

In practice, the pin 2 is shaped in a manner adequate for the correction to be performed, then slidingly receives the rings 5 in a number equal to the number of screws 4 to be installed; each ring 5 is then clipped in the cavity 10 of the corresponding screw 4, and the caps 15 are then positioned and tightened on the heads 11.

Tightening of the rings 5 by means of the lateral screws 14 and the inclined walls of the conical apertures 17 of the caps 15 allows a complete immobilization of the pin 2 with relation to the screw 4 to be ensured, by radial displacement of the rings 5, whatever the angular orientations of the rings 5 in the cavities 10 and the orientation of the pin 2 with relation to the screws 4.

It appears from the preceding that the invention provides a decisive improvement of the prior art by providing a vertebral arthrodesis device at once simple to manufacture and easy to position that ensures a complete immobilization of the pin in a given position.

It goes without saying that the invention is not limited to the embodiment described above by way of example but that it embraces on the contrary all variations of embodiments covered by the attached claims. Thus, said wall delimiting the cavity 10 may be flat at the bottom of this cavity, and not curved as previously described, the lateral zones of this wall keeping their aforesaid form of a segment of a hollow sphere.

1. A vertebral arthrodesis device comprising at least one pin designed to be positioned along the vertebrae that are to be immobilized, and parts for anchoring this pin to the vertebrae, each anchoring part presenting a head that delimits a cavity for receiving the pin and that receives the tightening means of the pin in said cavity, the assembly allowing the possibility of clearing the pin with relation to the anchoring part before tightening of the said tightening means, and the possibility of immobilizing this head with relation to this anchoring part in a given position, when this tightening is performed;
The device characterized in that:
The device comprises rings in a spherical form in a number equal to that of the anchoring parts, each ring presenting an inside diameter allowing its engagement slidingly on the pin and a plurality of slots distributed on its periphery, these slots extending between the outer surface of the ring and the said inside diameter and opening alternatively at one of the longitudinal openings of this inside diameter and at the other of these longitudinal openings;
The head of each anchoring part is shaped so that the said cavity that the head delimits may receive one of said rings with snap-on installation, and presents at least two lateral threaded holes opening from its proximal surface; and
Each anchoring part comprises a clamping ring shaped for positioning on the said head by overlapping the said ring, this clamping ring comprising lateral holes appropriate for coinciding with the threaded holes of the head and for receiving the tightening screws that can be screwed in these threaded holes, this clamping ring furthermore presenting a cone-shaped central aperture allowing the clamping ring to come into contact with the ring in the course of tightening of the said screws, so that this clamping ring tightens this ring between itself and the said head.

2. The device according to claim 1, characterized in that the head of each anchoring part comprises at least one slot opening in the bottom of the said cavity, giving the cavity a slight flexibility in a direction perpendicular to that according to which this cavity opens on the outside of the head.

3. The device according to claim 2, characterized in that the head of each anchoring part comprises two lateral slots opening in the bottom of the said cavity.

4. The device according to claim 3, characterized in that each slot is inclined in the direction of the other slot so that these slots converge in the direction of the bottom of the cavity.

5. The device according to claim 1, characterized in that the cavity of each anchoring part is delimited by a wall in the form of a hollow sphere segment presenting a diameter slightly less than that of each ring.

6. The device according to claim 5, characterized in that the cavity of each anchoring part is bordered by two lateral undercuts in the form of a segment of a hollow sphere, allowing, in the snap-on installation position of the ring, clearance of the pin with relation to the corresponding anchoring part.

7. The device according to claim 1, characterized in that the head of each anchoring part presents an enlarged form extending in a direction perpendicular to the direction according to which the cavity opens on the outside of the head, so that the form delimits two thick lateral walls, in the proximal surfaces of which said threaded holes are provided.
